(19) United States
(12) Patent Application Publication
(10) Pub. No.: US 2023/0363879 A1
(43) Pub. Date: Nov. 16, 2023

(54) SURGICAL IMPLANTS INCLUDING INDICIA

(71) Applicant: Covidien LP, Mansfield, MA (US)

(72) Inventors: Olesea Diaz-Chiosa, Naugatuck, CT (US); Russell V. Pribanic, Roxbury, CT (US)

(21) Appl. No.: 18/038,079

(22) PCT Filed: Dec. 17, 2021
(86) PCT No.: PCT/US2021/063974

§ 371 Date: May 22, 2023

Related U.S. Application Data
(60) Provisional application No. 63/131,852, filed on Dec. 30, 2020.

(51) Int. Cl. A61F 2/00

(52) U.S. Cl. CPC A61F 2/0063; A61F 2002/0072; A61F 2250/0097

(57) ABSTRACT

According to an aspect of the present disclosure a surgical system is provided. The surgical system includes a surgical implant and a camera. The surgical implant includes indicia that is non-visible to a human eye. The camera is configured to detect the indicia on the surgical implant.

### BACKGROUND

### Technical Field

The present disclosure relates to surgical implants including indicia. More specifically, the present disclosure relates to various types of surgical implants, including surgical mesh, sutures, tacks, clips, etc., which include fiducial demarcation for use in robotic surgical systems.

### Background of Related Art

Surgical mesh is commonly used to reinforce muscular walls when a weak area is detected and/or when a tissue herniation is observed. It can be ergonomically challenging, especially during inguinal and incisional procedures, to deploy and properly position mesh to tissue. Depending on the type of surgical procedure, location in the body, and surgeon preference, mesh may be secured to tissue using any combination of sutures, tacks, clips, staples, etc.

In addition to the challenge of properly positioning the mesh on tissue, it can also be a challenge to secure the mesh to tissue because of difficult access to the mesh, and the duration of the securing process, for instance. Suturing, for example, can be a time-consuming process, which may lead to surgical fatigue.

Suturing, applying tacks, and applying clips are now being accomplished by or with the assistance of robotic surgery. Accordingly, it may be helpful to include fiducial demarcation on the implant to be used as a reference frame to help guide a robotic system during the positioning and/or securing of mesh, for example.

### SUMMARY

The present disclosure relates to a surgical system including a surgical implant and a detecting device. The surgical implant includes indicia that is non-visible to a human eye. The detecting device is configured to detect the indicia on the surgical implant.

In disclosed embodiments, the indicia is made from indocyanine green.

It is further disclosed that the surgical implant includes secondary indicia that is visible to a human eye.

In aspects, the surgical implant is a surgical mesh, and the surgical system includes a suture or a surgical tack configured to secure the surgical mesh to tissue. It is disclosed that the suture or surgical tack includes indicia that is non-visible to a human eye. In embodiments, the indicia on the suture or surgical tack is made from indocyanine green.

In disclosed embodiments, the surgical implant is a surgical mesh, and the surgical system includes a surgical robot in communication with the detecting device and configured to secure the surgical mesh to tissue using suture or a surgical tack. It is disclosed that the suture or surgical tack includes indicia that is non-visible to a human eye.

It is also disclosed that the indicia is configured to be reflected by ultraviolet light.

The present disclosure also relates to a surgical implant including a first indicia, and a second indicia. The first indicia is made from a cyanine dye that is invisible to a human eye.

In disclosed embodiments, the second indicia is made from the cyanine dye that is invisible to a human eye.

It is also disclosed that the cyanine dye is indocyanine green.

In embodiments, the second indicia is visible to a human eye.

In aspects, the first indicia includes one of a linear pattern or a circular pattern.

It is disclosed that the cyanine dye is configured to be detected by a detecting device.

Further details and aspects of exemplary embodiments of the present disclosure are described in more detail below with reference to the appended figures.

### BRIEF DESCRIPTION OF THE DRAWINGS

Embodiments of the present disclosure are described herein with reference to the accompanying drawings, wherein:

FIG. 1 is a perspective view of a surgical mesh including a first type of fiducial demarcation, as seen by a device capable of detecting non-visible wavelengths;

FIG. 2 is a perspective view of a surgical mesh including a second type of fiducial demarcation, as seen by a device capable of detecting non-visible wavelengths;

FIG. 3 is a perspective view of the surgical mesh of FIG. 1 on tissue, as seen by a human eye;

FIG. 4A is a perspective view of the surgical mesh of FIG. 1 on tissue and a surgical robot capable of detecting non-visible wavelengths;

FIG. 4B is the area of detail depicted in FIG. 4A;

FIG. 5 is a perspective view of the surgical mesh of FIG. 4A secured to tissue with a suture and a portion of the surgical robot capable of detecting non-visible wavelengths;

FIG. 6 is a perspective view of the surgical mesh of FIG. 4A secured to tissue with tacks and a portion of the surgical robot capable of detecting non-visible wavelengths; and

FIG. 7 is a schematic illustration of a robotic surgical system configured for use in accordance with the present disclosure.

### DETAILED DESCRIPTION

Embodiments of the presently disclosed surgical implant are described in detail with reference to the drawings, in which like reference numerals designate identical or corresponding elements in each of the several views. Non-limiting examples of surgical devices according to the present disclosure include manual, robotic, mechanical and/or electromechanical surgical tack appliers (e.g., tackers), clip appliers, surgical forceps, and the like.

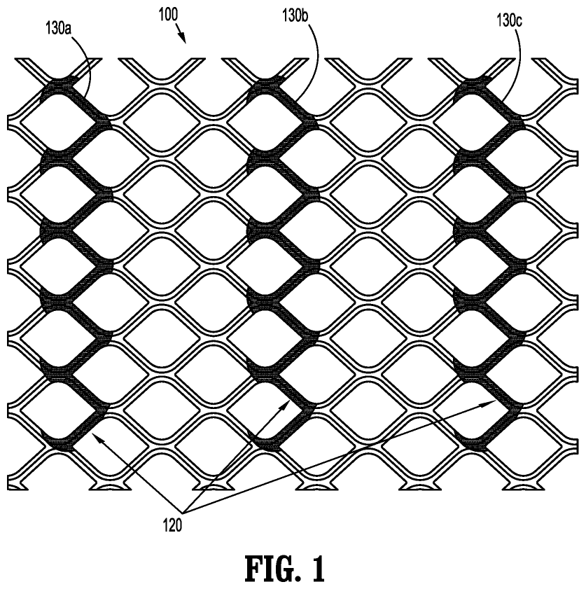

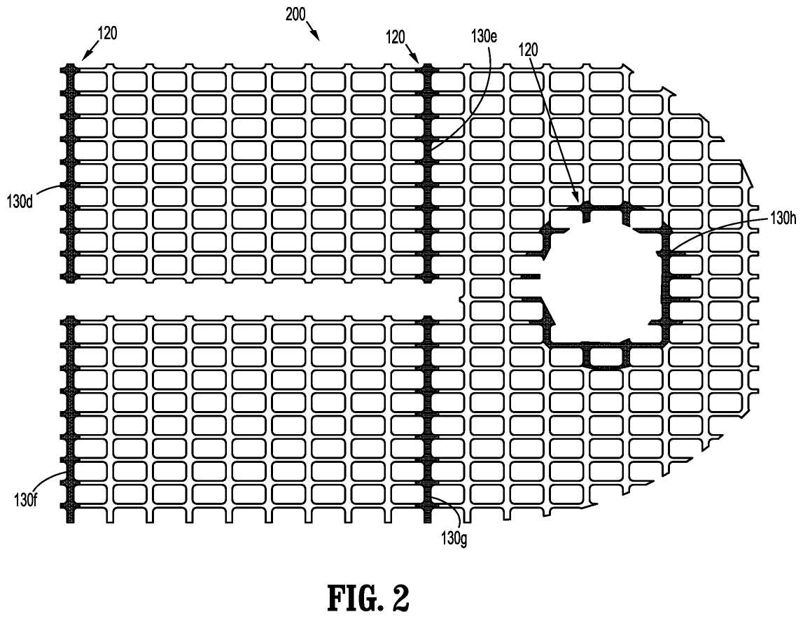

Referring initial to FIGS. 1-2, different embodiments of a surgical mesh are shown, and are generally designated as surgical mesh 100, 200, respectively. Surgical mesh 100, 200 is commonly used in surgical procedures to help reinforce tissue. The surgical mesh 100, 200 is placed over a void or a weak area of tissue, for instance, and is often secured into place using suture, tacks, clips, staples, etc. Once applied, the surgical mesh 100, 200 helps the tissue heal. After the tissue sufficiently heals, the surgical mesh 100, 200 and/or the suture, tacks, clips, staples, etc., may be surgically removed or may biodegrade within the patient's body after an appropriate amount of time.

Surgical robots are used to perform or assist with various surgical procedures to reduce the operating time, to reduce the possibility of surgeon fatigue, and to standardize such procedures. The surgical mesh 100, 200 of the present disclosure includes examples of fiducial demarcation or indicia that can be detected by surgical robots.

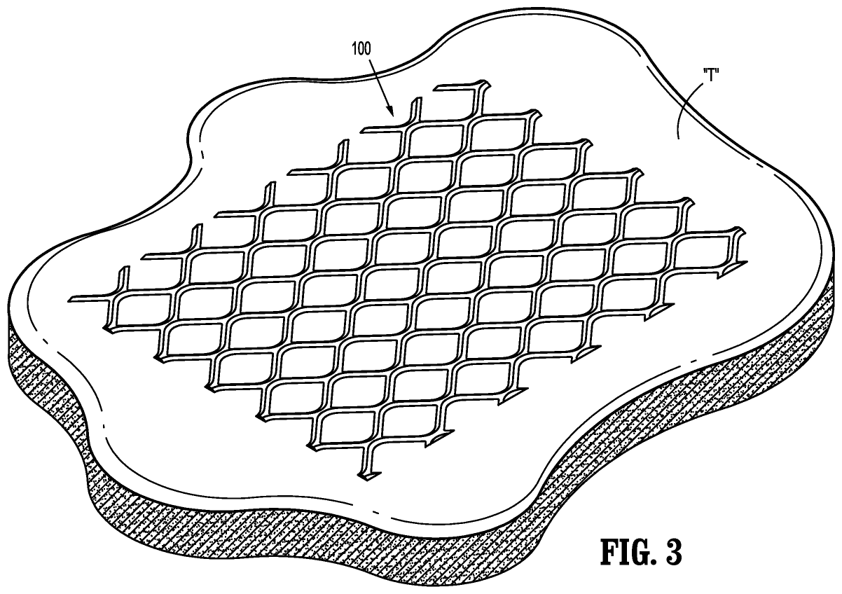

More particularly, the surgical mesh 100, 200 includes fiducial markers or indicia 120 (e.g., on the fibers of the mesh) that are configured to reflect non-visible (to the human eye) wavelengths of light. FIG. 3 depicts the surgical mesh 100 on tissue “T” without the aid of another tool to view or detect the indicia 120. Accordingly, the indicia 120 is not shown in FIG. 3. The indicia 120 may be coated onto the surgical mesh 100, 200 or impregnated into the surgical mesh 100, 200.

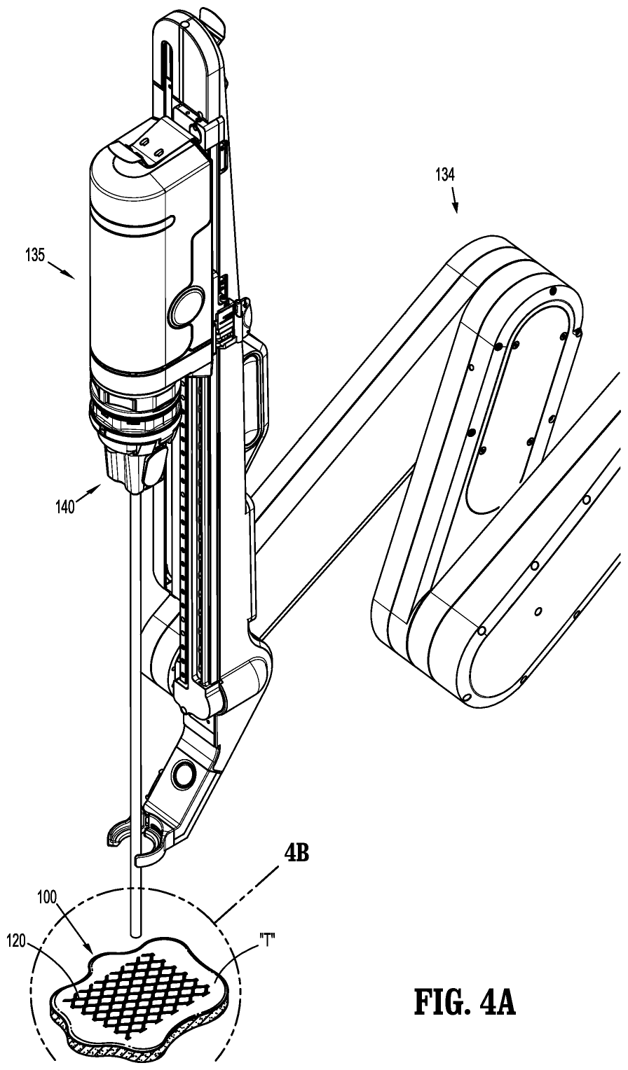

Referring now to FIG. 4A, a surgical robot 134 having a robotic arm 135 including a camera, filter, laser, detecting device, and/or ultraviolet light 140, for instance, is capable of reflecting the indicia 120 on the surgical mesh 100, thereby making the indicia 120 visible to the surgical robot 134, for example. Accordingly, FIG. 4B, which is an enlarged view of a portion of FIG. 4A, depicts what is visible to the surgical robot 134, including the indicia 120 on the surgical mesh 100.

The non-visible wavelengths of light reflected by the indicia 120 can be achieved by using a cyanine dye, e.g., Indocyanine Green (ICG). ICG is a fluorescent dye and is typically naturally eliminated from a patient's body in a relatively short amount of time (e.g., under one hour). Depending on the amount of solvent used and its concentration, the absorption and fluorescence spectrum of ICG is in the near-infrared region. ICG typically emits fluorescence between about 750 nanometers (nm) and about 950 nm. When ICG is used in medical applications (e.g., in blood plasma), its maximum absorption is about 800 nm. In the present disclosure, a surgical robot including a laser having a wavelength of about 780 nm can be used to detect the fluorescence of ICG on the indicia 120. The laser can be fine-tuned to filter out scattered light of the excitation beam, for instance.

Referring back to FIGS. 1 and 2, the indicia 120 on the surgical mesh 100, 200 includes several reference points, lines, or shapes 130a-130h. The reference points 130a-130h are customizable and can be designed for a particular type of surgical procedure and/or surgeon preference, for instance. The reference points 130a-130h may help enable robotic software to determine a frame of reference, and/or may help determine or guide a pathway or locations to emplace suture, tacks, etc.

With continued reference to FIGS. 1 and 2, two examples of different patterns of indica 120 are shown on the surgical mesh 100, 200. The different patterns of indica 120 can be used for various reasons. For instance, in FIG. 1, the indicia 120 on the surgical mesh 100 includes a first vertical line or linear pattern 130a, a second vertical line or linear pattern 130b, and a third vertical line or linear pattern 130c. With this pattern, it is envisioned that a surgeon guides the suturing pathway, and the robotic arm 135 of the surgical robot 134 is able to suture and fixate the surgical mesh 100 in a specified trajectory—for instance, from the first vertical line 130a, to the second vertical line 130b, and then to the third vertical line 130c.

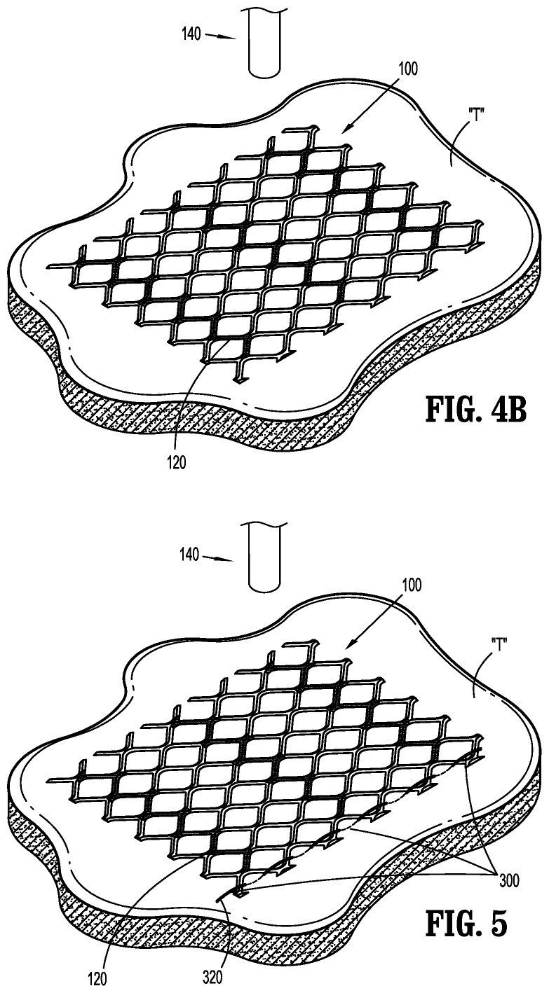

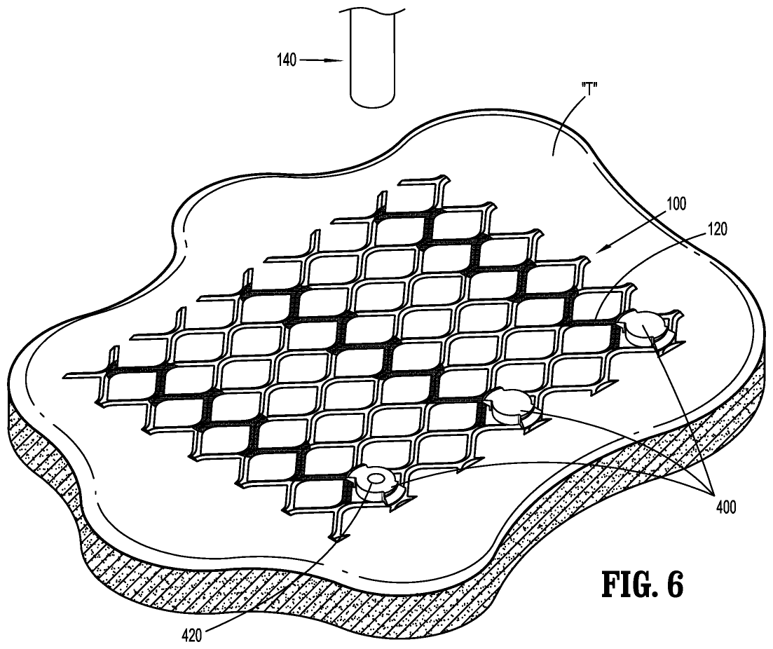

In FIG. 2, the surgical mesh 200 and indicia 120 are designed for a particular application and/or for positioning adjacent a particular anatomical location such as a major blood vessel, muscle or nerve in the inguinal region of a body. The indicia 120 of the surgical mesh 200 is configured to designate fixation points and to guide the surgical robot 134. For instance, the indicia 120 of the surgical mesh 200 includes four vertical lines or linear patterns 130d, 130e, 130f and 130g, which may denote boundaries for the placement of suture 300 (FIG. 5) and/or surgical tacks 400 (FIG. 6), and a circle or circular pattern 130h, which may denote designate a fixation point to a particular anatomical location. It is also envisioned that that some of the indicia 120 may be visible to the human eye, in addition to or as an alternative of being visible to the surgical robot 134 or detecting device.

FIG. 5 illustrates the surgical mesh 100 on tissue “T” with suture 300 helping to secure the surgical mesh 100 to the tissue “T.” FIG. 6 illustrates the surgical mesh 100 on tissue “T” with surgical tacks 400 helping to secure the surgical mesh 100 to the tissue “T.” Further, suture 300 (FIG. 5) and/or tacks 400 (FIG. 6) may include fiducial markers or indicia 320, 420, respectively. Similar to the indicia 120, the indicia 320, 420 may also be configured to reflect non-visible (to the human eye) waves of light. The robotic camera filter or ultraviolet light 140 that is used to reflect the indicia 120 on the surgical mesh 100, can also reflect the indicia 320, 420 on the suture 300 and tacks 400. Accordingly, the robotic software can use these indicia 320, 420 in connection with the indicia 120 of the surgical mesh 100 to further facilitate the proper positioning and pathway for emplacing the suture 300 and/or tacks 400. For instance, the robotic software can compare the suture 300 and/or tacks 400 that have already been embedded through the surgical mesh 100 with a pre-determined desired placement pattern or design of the suture 300 and/or the tacks 400.

While the surgical mesh 100, 200, the suture 300 and/or the tacks 400 may include fiducial markers or other indicia that is visible to the human eye, the inclusion of indicia 120, 320, 420 that is not visible to the human eye may be especially helpful during procedures that are at least partially performed by the surgical robot 134. Further, the surgical mesh 100, 200, the suture 300 and/or the tacks 400 may include both types of indicia: indicia that is visible to the human eye, and indicia 120, 320, 420 that is not visible to the human eye. More particularly, the inclusion of indicia visible to the human eye may assist a surgeon or operator in selecting the appropriate type of mesh, suture and/or tacks to be used for the surgical procedure. However, since the surgical mesh 100, 200, suture 300 and tacks 400 are quite small, it can be challenging to have both sets of indicia (including the indicia 120, 320, 420 that is configured to be detected by the surgical robot 134, and the indicia that is configured to be visible to the human eye) visible to the human eye without interfering with the user's ability properly decipher a particular indicia.

Surgical robots 134 and surgical meshes 100, 200 such as those described herein are configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

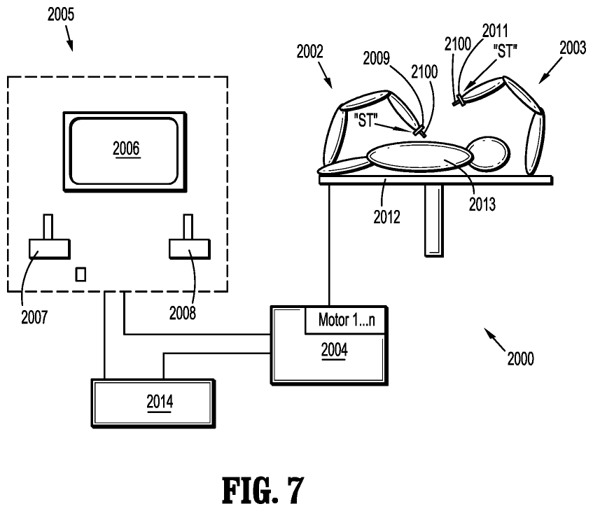

Referring to FIG. 7, a medical work station is shown generally as work station 2000 and generally may include a plurality of robot arms 2002, 2003; a control device 2004; and an operating console 2005 coupled with control device 2004. Operating console 2005 may include a display device 1006, which may be set up in particular to display three-dimensional images; and manual input devices 2007, 2008, by means of which a person (not shown), for example a surgeon, may be able to telemanipulate robot arms 2002, 2003 in a first operating mode.

Each of the robot arms 2002, 2003 may include a plurality of members, which are connected through joints, and an attaching device 2009, 2011, to which may be attached, for example, a surgical tool “ST” supporting an end effector 2100, in accordance with any one of several embodiments disclosed herein, as will be described in greater detail below.

Robot arms 2002, 2003 may be driven by electric drives (not shown) that are connected to control device 1004. Control device 2004 (e.g., a computer) may be set up to activate the drives, in particular by means of a computer program, in such a way that robot arms 2002, 2003, their attaching devices 2009, 2011 and thus the surgical tool (including end effector 2100) execute a desired movement according to a movement defined by means of manual input devices 2007, 2008. Control device 2004 may also be set up in such a way that it regulates the movement of robot arms 2002, 2003 and/or of the drives.

Medical work station 2000 may be configured for use on a patient 2013 lying on a patient table 2012 to be treated in a minimally invasive manner by means of end effector 2100. Medical work station 2000 may also include more than two robot arms 2002, 2003, the additional robot arms likewise being connected to control device 2004 and being telemanipulatable by means of operating console 1005. A medical instrument or surgical tool (including an end effector 2100) may also be attached to the additional robot arm. Medical work station 2000 may include a database 2014, in particular coupled to with control device 2004, in which are stored, for example, pre-operative data from patient/living being 2013 and/or anatomical atlases.

Reference is made herein to U.S. Pat. No. 8,828,023 to Neff et al., entitled “Medical Workstation,” the entire content of which is incorporated herein by reference, for a more detailed discussion of the construction and operation of an exemplary robotic surgical system.

Any of the components described herein may be fabricated from either metals, plastics, resins, composites or the like taking into consideration strength, durability, wearability, weight, resistance to corrosion, ease of manufacturing, cost of manufacturing, and the like.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. A surgical system, comprising:
a surgical implant including indicia that is non-visible to a human eye; and
a detecting device configured to detect the indicia on the surgical implant.

2. The surgical system according to claim 1, wherein the indicia is made from indocyanine green.

3. The surgical system according to claim 1, wherein the surgical implant includes secondary indicia that is visible to a human eye.

4. The surgical system according to claim 1, wherein the surgical implant is a surgical mesh, and wherein the surgical system further includes a suture configured to secure the surgical mesh to tissue.

5. The surgical system according to claim 4, wherein the suture includes indicia that is non-visible to a human eye.

6. The surgical system according to claim 5, where the indicia on the suture is made from indocyanine green.

7. The surgical system according to claim 1, wherein the surgical implant is a surgical mesh, and wherein the surgical system further includes a surgical tack configured to secure the surgical mesh to tissue.

8. The surgical system according to claim 7, wherein the surgical tack includes indicia that is non-visible to a human eye.

9. The surgical system according to claim 8, where the indicia on the surgical tack is made from indocyanine green.

10. The surgical system according to claim 1, wherein the surgical implant is a surgical mesh, and wherein the surgical system further includes a surgical robot in communication with the detecting device and configured to secure the surgical mesh to tissue using suture.

11. The surgical system according to claim 10, wherein the suture includes indicia that is non-visible to a human eye.

12. The surgical system according to claim 1, wherein the surgical implant is a surgical mesh, and wherein the surgical system further includes a surgical robot disposed in communication with the detecting device and configured to secure the surgical mesh to tissue using a surgical tack.

13. The surgical system according to claim 12, wherein the surgical tack includes indicia that is non-visible to a human eye.

14. The surgical system according to claim 1, wherein the indicia is configured to be reflected by ultraviolet light.

15. A surgical implant, comprising:
a first indicia made from a cyanine dye that is invisible to a human eye; and
a second indicia.

16. The surgical implant according to claim 15, wherein the second indicia is made from the cyanine dye that is invisible to a human eye.

17. The surgical implant according to claim 15, wherein the cyanine dye is indocyanine green.

18. The surgical implant according to claim 15, wherein the second indicia is visible to a human eye.

19. The surgical implant according to claim 15, wherein the first indicia includes one of a linear pattern or a circular pattern.

20. The surgical implant according to claim 15, wherein the cyanine dye is configured to be detected by a detecting device.
